(19) United States
(12) Patent Application Publication
(10) Pub. No.: US 2024/0278032 A1
(43) Pub. Date: Aug. 22, 2024

(54) FEEDBACK-CONTROLLED DEVICE FOR GEOMAGNETIC ION CYCLOTRON RESONANCE

(71) Applicant: Non Ionizing Radiation Personal Shielding S.R.L., Poggibonsi (IT)

(72) Inventor: Livio Giuliani, Roma (IT)

(21) Appl. No.: 18/571,247

(22) PCT Filed: Jun. 18, 2022
(86) PCT No.: PCT/IB2022/055646

§ 371 Date: Dec. 17, 2023

(30) Foreign Application Priority Data
Jun. 18, 2021 (IT) 102021000015971
Jun. 17, 2022 (WO) PCT/IB2022/055646

(51) Int. Cl. A61N 2/02; G16H 40/63

(52) U.S. Cl. CPC A61N 2/02; G16H 40/63

(57) ABSTRACT

A device for geomagnetic ion cyclotron resonance feedback-controlled by a variation in specific body impedance comprises an exposure system (2), a signal generator with an amplifier (3), an impedance meter (4), at least one gaussmeter (5) and a feedback circuit, which, guides the administration based on the specific body impedance (per single frequency) for a selected duration of time. The gaussmeter (5) generates a reference signal (R) representing an intensity value of a spatial distribution of the magnetostatic or magnetoquasistatic field, with a reading of components in three dimensions, inside the treatment volume (T). The signal generator with amplifier (3) is configured to control the coils (2a) by modulating one or more characteristics of the electromagnetic field as a function, simultaneously, of the impedance signal (I) received from the user's body, and the operating signal (C) with an operating signal at a frequency of between 100 kHz and 300 kHz.

### CROSS REFERENCE TO RELATED APPLICATIONS

The present application is a National Stage application under 35 U.S.C. § 371, of PCT Application No, PCT/IB2022/055646, filed Jun. 17, 2022, published as WO2022264104A1, which claims benefit of priority from Italian Patent Application No. 102021000015971, filed Jun. 18, 2021, the entirety of which are expressly incorporated herein by reference.

### FIELD OF THE INVENTION

The present invention relates to the technical field of devices for healthcare use.

In particular, the present invention relates to a device for ion cyclotron resonance feedback-controlled by a variation in specific body impedance which also takes account of the real contribution/interference generated by the geomagnetic field.

### BACKGROUND OF THE INVENTION

Ion cyclotron resonance has been recognized as an interaction between low-frequency electromagnetic fields and living matter following the reproduction in various countries of the so-called BLZ (Blackman-Liboff-Zhadin) effect.

In particular, an important biological reaction has been recognized with reference to the action of very low-frequency electromagnetic fields on capillary and transmembrane ion motion.

The application of this physicochemical effect to biology has led to surprising results in regenerative medicine through the stimulation of metabolism and cell differentiation and maturation in neuronal and pseudo-neuronal cells, normal or cancerous epithelial cells, cardiac cells, and also in bone and cartilage cells.

Ion cyclotron resonance appears to play an important role also in the prevention and treatment of degenerative diseases such as sporadic and familial Alzheimer's disease, as it shows an ability to reduce b-amyloid plaques in the brain and also an antibacterial action, making it possible to modulate biofilm production by antibiotic-resistant bacteria.

However, the known devices are still affected by defects and disadvantages that preclude an efficient use of this technology, despite the enormous nascent interest in the countless possible applications in the medical field.

The main problem with the known devices is that they are not generally built to exploit a known bioelectromagnetic interaction, but are rather built on an empirical basis, and are generally closed systems, with a pre-set list of operating conditions that is presented to the therapist, who can thus not freely choose the frequencies, waveforms or intensities to be applied, but rather chooses a set of pre-set values that in his or her opinion are the best suited to the symptoms determined in the patient.

Even the most advanced devices, such as those mentioned for the treatment of tumors, do not allow the individual operating parameters to be analyzed and selected precisely and accurately in order to optimize the therapeutic effect.

Furthermore, the known systems are not even configured to really operate under a condition of ion cyclotron resonance, as they are calibrated, designed and controlled on the basis of a fixed value for the geomagnetic field, thus without taking account of the variations therein, as is described, for example, in document WO2007/077532.

Given such imprecision, it is not actually possible to determine, in practice, any Liboff-Zhadin effect for any ion, thus making ion cyclotron resonance in fact inapplicable in the known devices.

### SUMMARY AND OBJECTS OF THE INVENTION

In this context, the technical task at the basis of the present invention is to propose a feedback-controlled device for ion cyclotron resonance that overcomes at least some of the aforementioned drawbacks of the prior art. In particular, it is an object of the present invention to provide a feedback-controlled device for ion cyclotron resonance that can be easily and efficiently configured to work optimally according to the specific situation in which it operates, in particular according to the ambient conditions and the conditions of the patient undergoing the ion resonance process.

The stated technical task and specified objects are substantially achieved by a feedback-controlled device for ion cyclotron resonance comprising the technical features disclosed in one or more of the appended claims. According to the present invention, there is shown a feedback-controlled device for ion cyclotron resonance which comprises an exposure system, a signal generator, an impedance meter and at least one gaussmeter.

The exposure system comprises a plurality of coils delimiting a treatment volume inside which a user can be at least partially accommodated and configured to generate an electromagnetic field inside the treatment volume.

The impedance meter is configured to record a specific impedance signal (i.e. one for each single frequency) representative of the impedance value at the extremities of the user's body when exposed to the electromagnetic field inside the treatment volume; this value depends on the frequency administered at that moment.

The gaussmeter is configured to generate a reference signal representative of an intensity value of a spatial distribution of the electromagnetic field inside the treatment volume and calculated also as a function of the magnetic inclination of that field.

The signal generator is configured to control the coils by modulating one or more characteristics of the electromagnetic field as a function, simultaneously, of the impedance signal and the reference signal, generating an operating signal at frequencies comprised between 100 and 300 kHz.

Advantageously, the device according to the present invention makes it possible to carry out a ion resonance treatment in an optimal, particularly efficient manner thanks to the real-time control of the electromagnetic field enabled by the continuous monitoring of the field distribution thereof and of the user's response to treatment through an assessment of his or her specific impedance at the various frequencies that can be delivered by the signal generator. The dependent claims, incorporated herein by reference, correspond to different embodiments of the invention.

### BRIEF DESCRIPTION OF THE DRAWINGS

Additional features and advantages of the present invention will become more apparent from the approximate, and thus non-limiting, description of a preferred but not exclusive embodiment of a feedback-controlled device for ion cyclotron resonance, as illustrated in the accompanying drawings, in which:

FIG. 1 shows a diagram representative of the main constituent elements of the device according to the present invention.

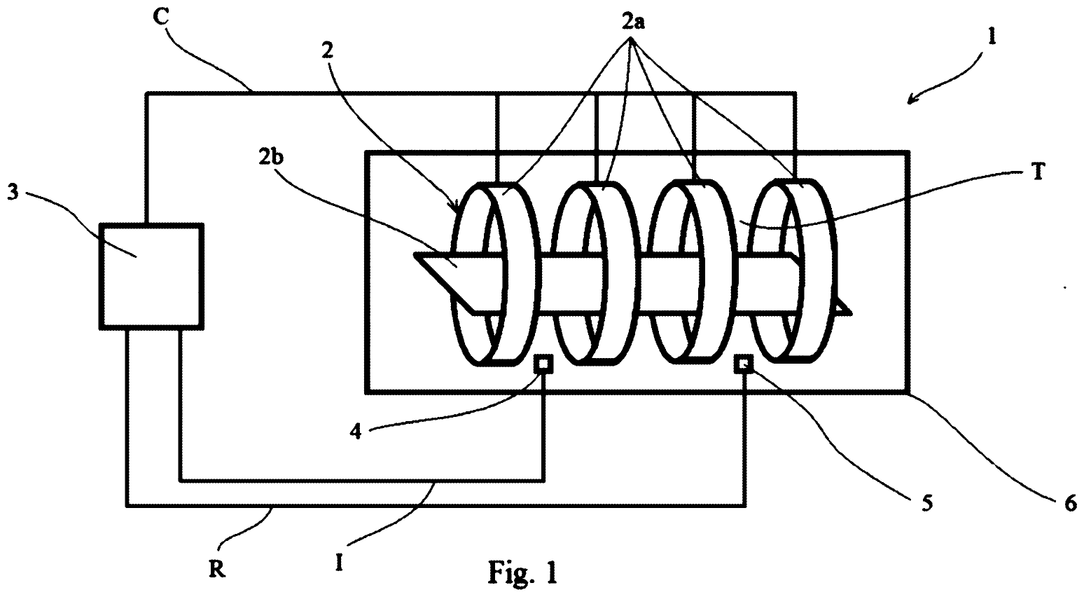

### DETAILED DESCRIPTION OF PREFERRED EMBODIMENTS OF THE INVENTION

In the appended FIGURES, the reference number 1 generically denotes a feedback-controlled device for ion cyclotron resonance, also identifiable and definable with the term “ion cyclotron”, which will be referred to hereinafter in the present description simply as the device 1.

More specifically, the device described herein is a device for the ion cyclotron resonance feedback-controlled by a variation in specific body impedance.

Ion cyclotron resonance is a phenomenon correlated with the movement of ions in a magnetic field and of a voltage signal delivered at an appropriate frequency.

Under the action of the magnetic field and the applied voltage the charged particles are accelerated according to a spiral motion.

Ion cyclotron resonance is not limited to the induction of cellular ionic currents of inorganic ions, but can be applied in the healthcare realm as well, since it can also be applied to organic molecules and is founded on a precise mechanism, whereby it is possible to induce in cells, also in vivo, ionic currents also originating from heavy ions and zwitterions, which exploit, for the formation and persistence thereof, the energy released by coherent clusters of water molecules present in cells.

These clusters, called coherence domains, can attract ions and release their electromagnetic energy so as to generate ionic currents in the solution, which have sufficient energy to overcome the potential barrier, as occurs for example in the Liboff-Zhadin effect.

Structurally, the device 1 comprises an exposure system 2, a signal generator 3, an impedance meter 4, at least one gaussmeter 5 and a feedback circuit.

The exposure system 2 comprises a plurality of coils 2a, delimiting a treatment volume “T”, in which a user can be at least partially accommodated, and configured to administer an electromagnetic field inside the treatment volume “T”

In particular, the coils 2a have respective inner surfaces that combine to define the treatment volume “T” adapted to at least partially accommodate the user therewithin.

In other words, the coils 2a have a substantially annular shape and dimensions such that their inside diameter is sufficiently large to permit a user to position him- or herself inside them; hence, the inner surface of the coils forms a portion of the wall of the treatment volume “T”.

In accordance with a first possible embodiment, the support 2b comprises a bed for the user, which the latter may lie upon during use of the device 1.

It may be observed that the term bed means any type of support on which the user may rest in a lying position, for example a hospital bed.

In this context, the plurality of coils 2a comprises a plurality of Helmholtz coils, preferably four Helmholtz coils.

In general, Helmholtz coils consist of identical circular magnetic coils that are placed symmetrically along a common axis and spaced equally apart so as to be evenly distributed relative to the treatment volume “T” they delimit.

Preferably, the Helmholtz coils are separated by a distance equal to the radius of the coils themselves; in this manner, the non-uniformity of the electromagnetic field at the center of the coils is minimized.

Preferably, there is a first Helmholtz coil associated with the head end of the bed and a second Helmholtz coil associated with the foot end thereof, with a further two Helmholtz coils optionally in an intermediate position and preferably movable axially (for example by means of a motor) between a non-operating position, in which they are brought alongside the first or second coil, respectively, so as to enable the user to lie down on the bed, and an operating position in which all coils are spaced equally apart from the adjacent coils.

In particular, the use of Helmholtz coils allows the interference of the geomagnetic field to be minimized, thus producing a region with a magnetic field intensity much closer to zero.

Further details regarding the specific structure and functionality of Helmholtz coils are specified in the following article:

Beiranvand, R. Analyzing the uniformity of the generated magnetic field by a practical one-dimensional Helmholtz coils system. Rev. Sci. Instrum. 2013, 84, 075109.

From an operational viewpoint, the Helmholtz coils are arranged coaxially along a main horizontal axis that extends along a plane in which the bed lies 2b.

In this manner, the bed 2b can be inserted or be insertable inside the treatment volume “T” by sliding the same along the main axis.

In other words, the Helmholtz coils define a substantially cylindrical treatment volume “T” in which the bed can be placed in a fixed or removable manner.

For example, the bed can be movable on horizontal guides allowing it to be slid into and out of the treatment volume “T”.

In this context, the device 1 preferably further comprises a compensation coil, which can likewise be made in the form of a Helmholtz coil, configured to compensate for the vertical component of the geomagnetic field.

In particular, the compensation coil is disposed perpendicularly relative to the other coils of the device 1, the Helmholtz coils thus usually being aligned with their central axis disposed horizontally, while the compensation coil is positioned in such a way as to have a central axis disposed vertically.

During use, it can also prove advantageous to position the bed 2b according to a north-south alignment that further enables the elimination of interference due to the horizontal component of the geomagnetic field.

In accordance with a further possible embodiment, the support 2b comprises a seat for the user, on which the latter can sit during use of the device 1.

In this context, the plurality of coils 2a comprises a plurality of Merritt coils; preferably it comprises three Merritt coils.

Further details regarding the specific structure and functionality of Merritt coils are indicated in the following article:

Merritt R, Purcell C and G Stroink (USA 1983). Uniform magnetic field produced by three, four, and five square coils. Review of Scientific Instruments 1983 March, 54:879; Epub Nov. 4 1998; DOI: 10.1063/1.1137480.

In particular, the Merritt coils are arranged coaxially along a main axis, preferably vertical; i.e. the Merritt coils are placed on top of one another so as to define a stack of coils extending vertically.

The seat is placed inside the treatment volume “T”, i.e. inside the space delimited by the Merritt coils.

In accordance with a further possible embodiment, the exposure system 2 comprises a booth for the user, inside which the latter can position him- or herself during use of the device 1.

A bed or a seat useful for supporting the user during operation of the device 1 can also be introduced inside the booth.

In this context, the plurality of coils 2a comprises a plurality of Cao coils. In particular, Cao coils represent a specific embodiment of Merritt coils, whose specific structure and functionality are indicated in the following article:

Qinjie Cao, Donghua Pan, Ji Li, Yinxi Jin, Zhiyin Sun, Shengxin Lin, Guijie Yang and Liyi Li (CHN 2018). Optimization of a Coil System for Generating Uniform Magnetic Fields inside a Cubic Magnetic Shield. Energies 2018, 11, 608

In general, and irrespective of the specific structural conformation of the exposure system 2, the device 1 comprises an impedance meter 4 configured to measure a specific impedance value of a user during use of the device 1, i.e. when the user is inside the treatment volume “T” and is exposed to the electromagnetic field generated by the coils 2a in accordance with the formula (1).

The impedance meter 4 is configured to record a specific impedance signal ‘Y at the user's body when it is exposed to the electromagnetic field, at an alternating current having the same frequency as is administered by the exposure system 2.

As will be explained in greater depth below, the specific impedance signal T will be used by the signal generator 3 to control the operation of the coils 2a.

The device 1 further comprises at least one gaussmeter 4 configured to measure an intensity value of a spatial distribution of the magnetostatic or magnetoquasistatic field with a reading of components in three dimensions, in particular inside the treatment volume “T”, and to generate a reference signal “R” representative of that intensity value.

In particular, the gaussmeter 5 is capable of providing the three spatial coordinates of the geomagnetic field in the treatment volume “T” and is connected or connectible to a circuit that calculates the mean and variance of the reference signal “R” over time.

In this manner it is possible to take into account, in a particularly precise and accurate manner, the actual real value of the magnetostatic field in the place and at the moment in which the device 1 is activated.

In order to better understand the significance of this innovation compared to what is offered by the prior art, let us consider the formula that allows a determination of the ion cyclotron resonance frequency, once the static field is known, for the ion cyclotron resonance: 2πv=qB_{O}/m, where v is the desired frequency and B₀ is the modulus of the static field parallel to the variable field.

Let us consider the divalent calcium ion. If we assume B₀=47.8 mT−the value of the geomagnetic field measured by the Observatory of Castel Telesino (TN) on 29 Dec. 2019—the ion cyclotron resonance frequency of the calcium would be equal to 36.37 Hz.

However, by measuring the field with a reading of the components thereof in the three direction it is possible to take account of the magnetic inclination lo in order to determine the real, actual horizontal component, which will be coupled to the variable magnetic field that will be administered, thus B₀=47.8*cos(62.38°), leading to an ion cyclotron resonance frequency of the calcium equal to 16.86 Hz.

More in general, the contribution to the determination of the correct operating frequency of the device 1 given by the geomagnetic field is calculated on the basis of measurements gathered from the impedance meter applying the formula 2πv=(qB₀/m) sin l₀ if the variable magnetic field with a frequency v is in the direction of the zenith (vertical) of the measuring point and 2πv=(qB₀/m) sin l₀ if the variable magnetic field with a frequency vis tangent to the Earth's surface (horizontal) in the measuring point; where, as indicated above, l₀ is the magnetic inclination in the point where the device 1 is used.

In other words, the reference signal is generated taking into account not only the intensity of the magnetostatic field, in particular of the geomagnetic field, but also the specific magnetic inclination thereof, thus making it possible to obtain a precise measurement that leads to the determination of the correct ion cyclotron resonance frequency. As will be explained in greater depth below, the reference signal “R” will also be used by the signal generator 3 to control the operation of the coils 2a.

Preferably, the at least one gaussmeter 5 is a precision gaussmeter, that is, it has a high sensitivity, preferably equal to or less than 25 nanoTesla (nT), i.e. it is capable of identifying and discriminating variations in the magnetic field equal to or less than 25 nT.

The signal generator 3, on the other hand, is configured to control the plurality of coils 2a, thereby enabling one or more characteristics of the electromagnetic field to be modulated on the basis of the specific impedance signal T and/or the reference signal “R”.

In other words, the signal generator receives the specific impedance signal ‘Y from the impedance meter 4 and the reference signal “R” from the at least one gaussmeter 5 and on the basis of those signals controls and governs the operation of the coils 2a so as to vary the electromagnetic field generated by the latter.

In particular, the signal generator 3 is configured to generate an operating signal “C”, which can be either a single magnetic signal at an ion cyclotron resonance frequency for stimulating the formation of a current of ions in tune with the frequency itself; or a double signal, the same one with the addition of a cyclotron frequency of one of the hydroxonium hydrates, in order to increase the conductivity of the intracellular water or a carrier radio signal having a frequency of between 100 and 300 kHz and which can preferably have a sinusoidal or triangular or square or sawtooth waveform, with at least two modulating waves with distinct ion cyclotron frequencies, by means of which it is possible to control the plurality of coils 2a.

Advantageously, the signal generator 3 can be further configured to generate a pre-exposure signal suitable for controlling the operation of the coils 2a at the ion cyclotron resonance frequency of hydroxonium and/or of one of the hydrates thereof (for example the Zundel cation or an Eigencation).

The device 1 can thus be operated under two regimes: a regime controlled by the operating signal “C” and a regime controlled by the pre-exposure signal.

By controlling the operation of the device 1 in a first moment by means of the pre-exposure signal it is possible both to prolong the impact of the Zhadin effect on the ions and protonate any water that may be present in the tissue/sample on which the device 1 is being used, so that the signal/noise ratio of the ionic currents induced by the device 1 itself will be improved when subsequently controlled with the operating signal “C”, thus enhancing the overall performance.

The signal generator 3 can also be configured to generate an operating signal “C” that comprises an amplitude-modulated carrier wave and has a frequency comprised between 100 and 300 kHz, by means of which it is possible to modulate the electromagnetic field by controlling the plurality of coils 2a.

In particular, a greater biological effectiveness has been observed in exposure to a low- or very low-frequency amplitude-modulated carrier wave rather than directly to low-frequency waves, as discussed for example in the following publications:
Bawin S M, Adey W R, Sabbot I M (USA 1978). Ionic factors in release of 45Ca2+ from chicken cerebral tissue by electromagnetic fields. PNAS 1978 December; 75(12):6314-8;
Lyle D B, Schechter P, Adey W R, Lundak R L. Suppression of T-lymphocyte cytotoxicity following exposure to sinusoidally amplitude-modulated fields. Bioelectromagnetics. 1983; 4(3):281-92;
Blackman C F, Benane S G, House D E, Joines W T (USA 1985). Effects of ELF (1-120 Hz) and modulated (50 Hz) R F fields on the efflux of calcium ions from brain tissue in vitro. Bioelectromagnetics. 1985; 6(1):1-11;
Vignati M, Giuliani L. radiofrequency near high voltage power lines. Environ Health Persp 105(S6):1535.

From an operational viewpoint, the operating signal “C” is generated as a function at least of the reference signal “R” and the specific impedance signal ‘Y so as to reproduce, in particular, the frequencies at which the user has shown a higher relative specific impedance (i.e. in relation to a specific physiological impedance value), or variation in specific impedance.

This operation is managed by the feedback circuit, which records the variations in specific impedance at the user's body, both of the resistive component and of the reactive one and their relationship, thereby determining, in the event of a variation in the latter parameters beyond certain thresholds that can be programmed, a trigger signal for the signal generator, for the delivery of the magnetic field at the frequency that has brought about the parametric variation beyond the threshold, for a time that is likewise programmable.

In other words, the device 1 has a closed-loop control system in which the spatial distribution of the electromagnetic field inside the treatment volume “T” and the specific impedance of the user are fed back and used to vary the operating signal “C” so as to promptly adapt the coils 2a to generate an electromagnetic field such as to maintain and maximize the efficiency of the ion cyclotron resonance effect.

In particular, the operating signal “C” is varied in such a way as to maintain the device 1 in an operating regime in which the specific impedance signal “I” is maximized.

The determination of the frequency at which the user shows the greatest relative impedance can be made during a preliminary calibration in a global manner, with reference solely to a continuous test exposure current; or local, with a scan that subjects the patient to brief exposures at every selectable frequency, so brief as to cause negligible disturbance, for example in the order of a second.

In other words, it is possible to generate a continuous operating signal “C” to measure the user's response to exposure to the electromagnetic field generated by the coils 2a in general terms, or else perform a progressive scan, exposing the user to all the frequencies that can be generated by the signal generator 3 and accurately mapping the impedance value manifested by the user at every single frequency used.

In this manner, during use of the device 1, the impedance meter 4 will generate the specific impedance signal T and the signal generator 3 will compare the new measurements of specific impedance with the values measured in a global or local manner during calibration of the device 1.

In the former case, the comparison will be direct and if for one frequency the ratio between the new measurement and the old one is identical, disregarding the systemic and random errors of the measurement, the patient will be exposed to that frequency for a time and at an intensity defined by the therapist and in any case sufficient to obtain on the user all the beneficial effects deriving from the ion resonance effect.

Ordinarily, exposure is maintained at about ten minutes or a thousand seconds; the intensity will ordinarily be in order of tens and hundreds of micro Tesla or nano Tesla.

The device 1 thus makes it possible to have a real-time control of the effectiveness of the therapeutic action thanks to the continuous readings of the impedance values and spatial distribution of the electromagnetic field generated respectively by the impedance meter and the at least one gaussmeter, further enabling them to be compared with the calibration values in order to determine the operating regime at which the user manifests the maximum response.

The device 1 can further comprise a plurality of sensors configured to detect a respective plurality of physiological parameters and generate a physiological signal representative of at least one of those physiological parameters.

By way of non-limiting example, the plurality of sensors comprises at least one of: a blood pressure sensor, a blood oxygenation sensor and a blood glucose detector. Advantageously, the signal generator 3 is further configured to control the plurality of coils 2a by modulating one or more characteristics of the electromagnetic field as a function also of the physiological signal considered individually or evaluated in combination with the reference signal “R” and specific impedance signal “I”.

In particular, the physiological signal could detect a condition of excessive stress of the user during the use of the device 1 and thus bring about an immediate interruption of the procedure (for example by blocking the generation of the operating signal “C”) or generate an error/alert signal of an audible and/or optical type to alert an operator of the occurrence of a situation of discomfort or potential danger to the user's health.

Furthermore, the device 1 can comprise at least one protective casing 6 disposed around the plurality of coils 2a and cooperating therewith to define the treatment volume “T”.

In other words, the protective casing 6 can define a physical wall for the treatment volume “T” which serves to cover in particular the portions of the wall of the treatment volume “T” left free by the coils 2a.

The protective casing 6 is configured to shield the treatment volume “T” from the geomagnetic field, thus preventing the latter from disturbing the electromagnetic field and enabling it to be controlled more precisely and efficiently.

In particular, the protective casing can comprise and/or be made of a metallic glass material.

The term metallic glass means a solid metal, usually an alloy, with an atomic structure that is amorphous, hence not crystalline.

In particular, according to a preferred embodiment, the metallic glass comprises a ternary metallic glass alloy based on iron or nickel added to boron in vitreous form and added to yttrium, for example according to the formula Y₄Fe₇₅B₂₁.

The alloy identified above is characterized by a coercivity of around 40 A/m and a specific electric conductivity greater than 1/90. In accordance with possible alternative embodiments, the yttrium can be replaced with an element that has an atomic size of not less than 130% of the size of iron and whose percentage in the alloy is no less than 3% or greater than 10%. The percentage of boron can also vary, between 18 and 27%.

As indicated above, the iron can be replaced at least partially by nickel or, according to possible alternatives that will be discussed in greater detail below, also by structures comprising palladium and/or zirconium in order to make the protective casing 6 from metallic glass material.

In general, the protective casing 6 can be made according to the parametric formula:

Y₃₊ₐFe_{79-a-b-c}Nl_{b}B_{18+c} wherein the parameters indicated can take on the following values:
a is comprised between 0 and 3 in atomic percentage;
b is equal to 0 or 67 in atomic percentage;
c is comprised between 0 and 9 in atomic percentage.

If a=3, b=67, c=9 the iron is completely replaced in the alloy by nickel.

A greater percentage of boron renders the alloy glassier, whereas a greater percentage of yttrium makes it possible to compensate for the increased molecular weight of the alloy due to the substitution of nickel for iron.

The specific conductivity of the alloy as parameterized above favors the effect of shielding the geomagnetic field.

The use of a protective casing 6 made of a metallic glass material has the further advantage of reducing the risk of occurrence of a feeling of claustrophobia in the user, since it is a material that is transparent to visible light.

Alternatively or additionally, the protective casing 6 can be made of and/or comprise a protective foil (preferably two protective foils, one on top of the other) made of mu-metal.

In accordance with a preferred embodiment, the mu-metal comprises Cu₅Ni₇₇Fe₁₆Cr₂ or Co₂₅₈. By way of example, a 0.35 mm double layer of permalloy (Ni₅₀₊ₓFe₅₀₋ₓ) or mu-metal would induce, in the case of an industrial electromagnetic field (50/60 Hz), a core loss (substitute for the specific absorption rate for radiofrequencies) of up to 0.2 mW/Kg, i.e. 0.2 μW/g.

Compared to the SAR limit introduced in Europe for partial exposure in the head area, namely, 2 μW/g (also adopted in China; in the USA and Japan it is 20% lower), it would be 10,000 times lower.

Since the performances of such alloys are even better with reference to radiofrequencies and a static magnetic field, this shielding represents the ideal solution for enabling ion cyclotron resonance treatments, given that, in the absence of a geomagnetic field, the system can administer a known static magnetic field with an error of less than a nanoTesla and consequently alternating magnetic fields, including Schumann waves, with equally precise frequencies.

In general it is worth observing that it is advantageously possible also to subtract the self-induced component of the coil from the intensity of the static field component in order to obtain an even more precise measurement.

Furthermore, the device 1 can further comprise a lighting system, for example an LED lighting system.

The lighting system is preferably powered by direct current in order not to cause electromagnetic interference.

In particular, the working frequency of the device 1 is determined also taking into account the static magnetic field induced by the lighting system. Advantageously, the use of the present device 1 permits important applications in the treatment of eukaryotic and prokaryotic cells (and viruses) endowed with pseudovilli extruded above the cell membrane (envelope), with the partial destructuring of the cellular cytoskeleton, due to electromagnetic resonance of actin at 50 Hz, which causes the collapse of pseudomicrovilli within the cytoskeleton.

For example, in the case of the coronavirus the mechanism of infection has been identified as the spike protein (S), consisting of two domains (S1-S2), as in SARS1-CoVid19, and its binding function resides in the S1 domain of the spike protein itself, whereas the molecule that lyses the membrane of the host cell and permits the penetration of the virus resides in the S2 domain.

In SARS2-Covid19 the S1 domain is activated by the enzyme furin, as by the ACE2 receptor of the host cell, a receptor analogous to the ACE receptor of SARS1-CoVid19.

In particular, the spike protein rests, outside the viral envelope, on an actin-like filament that could be made to collapse by exposure to a frequency of 50 Hz or higher, within 100 Hz, with an amplitude in the order of a milliTesla, as the actin filaments of eukaryotic cells collapse under such exposure. Sec, among many others, S Rieti, V Manni, A Lisi, L Giuliani, et al. SNOM and AFM microscopy techniques to study the effect of non-ionizing radiation on the morphological and biochemical properties of human keratinocytes cell line (HaCaT). J Microsc 213: Pt 1. 20-28 2004 Jan.

In such a case the spike proteins of the coronavirus would all collapse together, ending up in the viral envelope (the physical interactions of sufficient amplitude affect the whole target population at the same instant); and would no longer be able to bind the furin or ACE2 of any host cell.

The effect would have a duration of about one hour and would in any case persist throughout the whole time of exposure, which could be continuous, since exposure to an industrial magnetic field at a few milliTesla is comparable to the exposure limit for workers valid in Italy and other European countries and the possible use of higher exposure values would nonetheless, for the purposes of the present invention, fall within the upper limits (6 mT at the industrial frequency), for the compliance with which earthing of the user's bed or seat and the equipotential bonding thereof would be sufficient.

The therapy implementable by means of the present device 1 aimed at destructuring the pseudo actin filaments of the virus would be effective in particular in the initial stage of infection, i.e. the stage of viral attack.

Any contraindication due to a lower mobility of B lymphocytes could be modulated with an exposure aimed at target organs, also through an exposure probe, in cases of intubation, which only marginally involves circulating blood.

And on the other hand, if the exposure should favor the release of angiotensin, that could contribute to dampening the inflammatory effect of the cytokine storm caused by the virus and would be synergistic with treatment with heparin, for which the therapeutic treatment at high doses has been authorized, on an experimental basis, by the Italian Medicines Agency (AIFA).

The treatment of CoVID19, as well as of SARS, at a severe and critical stage could be supported by exposure to ELF/SLF, given its inhibitory action on interleukin 6, which presumably causes the cytokine storm triggered by the coronavirus.

The same ELF/SLF treatment can be adopted with other coronaviruses, the influenza virus, with picoRNA viruses in general and in diseases caused by viruses that carry binding proteins on extruded filaments of the envelope.

In particular, the two parts S1 and S2 of the spike protein S are mutually mobile and establish between them a potential difference of up to 0.5 V in the binding phase. See, among many others, Cho Y-K, Y-w, Frank M et al. Structure, Dynamics, Receptor Binding, and Antibody Binding of the Fully Glycosylated Full-Length SARS-COV-2 Spike Protein in a Viral Membrane. J Chem Theory and Comp 2021 17(4):2479-87, doi: 10.1021/acs.jctc.0c01144.

The frequencies between 100 and 200 kHz (indicated as preferential for the device described herein) are capable of altering the charge distribution of the S1 and S2 complexes of the spike protein, preventing the ‘jaw-like’ functioning whereby the spike protein attaches to the ACE2 receptor with the S1 complex while it lyses the host cell membrane by closing the S2 complex over the attachment point. Similarly, it has been shown that in the electromagnetic treatment of brain tumors long radio waves interfere with the arrangement of homologous charges that form along the parallels of the mitotic spindle of tumor cells during reproduction; as said homologous charges become denser, the spindle dilates until mitosis is reached.

In the same manner, the frequencies indicated would alter the distribution of charges between the S1-S2 subsets of the spike protein, preventing the functioning of the two subsets, above all of S2 in the lysis of the host cell membrane in order to pour viral material inside it.

The device 1 in fact makes it possible to apply a mechanism interfering with the virus SARS-2CoV-19 and to couple, with that basic mechanism, the dual ion cyclotron mechanism due to the double modulators that the device enables control of in the complex of coils 2a.

Advantageously, the present invention achieves the proposed objects, overcoming the aforementioned drawbacks of the prior art by providing the user with a device for ion cyclotron resonance that is particularly versatile and capable of operating under every condition and responding rapidly and efficiently to the conditions of the user during the treatment.

What is claimed is:

1. A device for geomagnetic ion cyclotron resonance feedback-controlled by a variation in specific body impedance of a body of a user, comprising:
an exposure system (2) comprising a plurality of coils (2a) having respective inner surfaces defining a treatment volume (T) adapted to at least partially accommodate the user and configured to administer an electromagnetic field having a frequency in the treatment volume (T);
an impedance meter (4) configured to measure a specific impedance value of a user when exposed to the electromagnetic field inside the treatment volume (T) and to generate a specific impedance signal (I) determined by an alternating current having the same frequency as the electromagnetic field administered by the exposure system (2) corresponding to an impedance value specific to the body of the user;
at least one gaussmeter (5) configured to measure an intensity value of a spatial distribution of a magnetostatic or magnetoquasistatic field with a reading of components in three dimensions inside the treatment volume (T) and to generate a reference signal (R) representative of the intensity value calculated as a function of a magnetic inclination of the magnetostatic or magnetoquasistatic field;
a signal generator (3) configured to control the plurality of coils (2a) by modulating one or more characteristics of the electromagnetic field by means of an operating signal (C) having a frequency between 100 kHz and 300 kHz generated as a function, simultaneously, of the specific impedance signal (I) and the reference signal (R);
a feedback circuit configured to receive variations in the impedance value, both in a resistive component and in a reactive component, as well as in a ratio between the reactive component and the resistive component, and to selectively produce a trigger signal for the signal generator, in the event of an exceeding of the relative specific impedance or a variation beyond a predefined threshold of the impedance, the trigger signal being configured to activate, for a predefined time interval, a delivery of the electromagnetic field at the frequency.

2. The device according to claim 1, comprising a plurality of sensors configured to detect a respective plurality of physiological parameters and to generate a physiological signal representative of at least one of the physiological parameters, the plurality of sensors comprising at least one of: a blood pressure sensor, a blood oxygenation sensor and a blood glucose detector.

3. The device according to claim 2, wherein the signal generator (3) is further configured to control the plurality of coils (2a) by modulating one or more characteristics of the electromagnetic field as a function also of the physiological signal and of the specific impedance signal (I).

4. The device according to claim 1, wherein the signal generator (3) is configured to generate the operating signal (C) having an amplitude-modulated waveform, with at least two modulating waves with distinct ion cyclotron frequencies controlling the plurality of coils (2a).

5. The device according to claim 1, wherein the at least one gaussmeter (5) is a precision gaussmeter (5) having a sensitivity equal to or less than 25 nT.

6. The device according to claim 1, wherein the exposure system (2) comprises a bed for the user, and the plurality of coils comprise a plurality of Helmholtz coils (2a), arranged coaxially along a main horizontal axis of the bed, the bed being insertable in the treatment volume (T) along the main axis.

7. The device according to claim 1, wherein the exposure system (2) comprises a seat for the user and the plurality of coils comprise a plurality of Merritt coils (2a), arranged coaxially along a vertical main axis, the seat being inserted in the treatment volume (T).

8. The device according to claim 1, wherein the exposure system (2) comprises a booth for the user and the plurality of coils comprise a plurality of Cao coils (2a) coupled to the booth so as to define the treatment volume (T) in cooperation with the booth.

9. The device according to claim 1, further comprising at least one protective casing (6) disposed around the plurality of coils (2a) and cooperating to define the treatment volume (T), the protective casing (6) being configured to shield the treatment volume (T) from a geomagnetic field.

10. The device according to claim 9, wherein the protective casing (6) comprises a metallic glass material, the metallic glass material preferably comprising Yr₃₊ₐFe_{39+a-b-c}Ni_{40+b}B_{18+c}, wherein:
a is between 0 and 3 in atomic percentage;
b is equal to 0 or 67 in atomic percentage; and
c is between 0 and 9 in atomic percentage.

11. The device according to claim 9, wherein the protective casing (6) comprises at least one protective foil, the protective foil comprising a mu-metal.

12. The device according to claim 1, further comprising a light emitting diode (LED) lighting system, the lighting system being configured to be powered with direct current.

13. A method for geomagnetic ion cyclotron resonance feedback-controlled by a variation in specific body impedance of a body of a user, comprising:
administering an electromagnetic field at a frequency to the body of the user to user within a treatment volume (T) with an exposure system (2) comprising a plurality of coils (2a);
measuring a specific impedance value (I) of a user when exposed to the electromagnetic field with an impedance meter (4) based on an alternating current having the same frequency as the electromagnetic field, corresponding to an impedance value specific to the body of the user;
measuring an intensity value of a spatial distribution of a magnetostatic or magnetoquasistatic field with a magnetic of components in three dimensions inside the treatment volume (T) with at least one gaussmeter (5), to generate a reference signal (R) representative of the intensity value calculated as a function of a magnetic inclination of the magnetostatic or magnetoquasistatic field;
automatically controlling the plurality of coils (2a) by modulating one or more characteristics of the electromagnetic field by means of an operating signal (C) having a frequency between 100 kHz and 300 kHz generated as a function, simultaneously, of the specific impedance signal (I) and the reference signal (R);
selectively producing a trigger signal for the signal generator, in the event of an exceeding of the relative specific impedance or a variation beyond a predefined threshold of the impedance, using feedback dependent on variations in the impedance value, both in a resistive component and in a reactive component, as well as in a ratio between the reactive component and the resistive component, the trigger signal being configured to activate, for a predefined time interval, a delivery of the electromagnetic field at the frequency.

14. The method according to claim 13, further comprising detecting a respective plurality of physiological parameters with a plurality of sensors, and generating a physiological signal representative of at least one of the physiological parameters, the plurality of sensors comprising at least one of: a blood pressure sensor, a blood oxygenation sensor and a blood glucose detector.

15. The method according to claim 14, further comprising controlling the plurality of coils (2a) by modulating one or more characteristics of the electromagnetic field as a function also of the physiological signal and of the specific impedance signal (I).

16. The method according to claim 14, further comprising generating the operating signal (C) having an amplitude-modulated waveform, with at least two modulating waves with distinct ion cyclotron frequencies controlling the plurality of coils (2a).

17. The method according to claim 14, wherein the at least one gaussmeter (5) is a precision gaussmeter (5) having a sensitivity equal to or less than 25 nT.

18. An ion cyclotron resonance feedback-controlled treatment system, comprising:
a plurality of coils configured to generate an oscillating electromagnetic field having a frequency surrounding a body of a user;
an impedance measurement subsystem configured to measure a specific impedance value of body of the user at the frequency both in a resistive component and in a reactive component, as well as in a ratio between the reactive component and the resistive component;
a magnetometer configured to quantitatively measure a three dimensional magnetic inclination of a magnetic field surrounding the body of the user and produce a reference signal dependent thereon;
a feedback dependent signal generator configured to control the plurality of coils to modulate at least one characteristic of the electromagnetic field dependent on both the specific impedance signal and the reference signal; and
an automated control, configured to activate the signal generator for a period, dependent on a value of the specific impedance value.

19. The system according to claim 18, further comprising a sensor configured to determine a physiological parameter, selective from the group consisting of a blood pressure, a blood oxygenation and a blood glucose.

20. The system according to claim 19, wherein the feedback dependent signal generator configured is further dependent on the physiological parameter.
